(19) United States
(12) Patent Application Publication
(10) Pub. No.: US 2013/0309215 A1
(43) Pub. Date: Nov. 21, 2013

(54) PHARMACEUTICAL COMPOSITION

(71) Applicant: Bernd G. SEIGFRIED, Limburgerhof (DE)

(72) Inventor: Bernd G. SEIGFRIED, Limburgerhof (DE)

(73) Assignee: MIKA Pharma Gesellschaft fur die Entwicklung und Vermarktung pharmazeutischer Produkte mbH, Speyer (DE)

(21) Appl. No.: 13/571,799

(22) Filed: Aug. 10, 2012

(30) Foreign Application Priority Data
May 15, 2012 (DE) 10 2012 009 575.9

(51) Int. Cl. A61K 47/44; A61P 37/08; A61P 17/00; A61P 25/00; A61P 31/00; A61K 31/49; A61P 31/10; A61K 31/454; A61K 31/405; A61K 31/557; A61P 29/00; A61P 5/26; A61P 5/28; A61K 31/56; A61P 25/08; A61P 7/02; A61K 38/43; A61K 31/7105; A61K 31/711; A61K 31/713; A61P 17/04; A61P 3/10; A61P 31/12; A61P 37/06; A61P 17/12; A61P 17/02; A61K 36/00; A61P 25/18; A61P 25/20; A61P 21/02; A61P 1/08; A61P 33/00; A61P 1/16; A61P 9/00; A61P 11/10; A61P 31/04; A61P 11/14; A61P 11/06; A61P 7/10; A61P 37/02; A61K 31/196; A61K 31/192; A61P 25/16; A61P 3/02; A61P 23/02

(52) U.S. Cl. 424/94.1; 514/785; 514/304; 514/323; 514/415; 514/169; 514/44 R; 424/725; 514/567; 514/570; 514/784

(57) ABSTRACT

A pharmaceutical composition for application in human and animals, with at least one systemically and/or locally acting, topically applicable active ingredient and with at least one phospholipid, improving the transport of the active ingredient trough the cell membrane and containing a concentration of at least 60% by weight phosphatidylcholine, referring to the phospholipid, is described. The composition shows such a liquid consistency, that it is able to be sprayed as droplets or as a foam, whereas in the composition such a phospholipid is included, that additionally contains oil in a concentration of maximum 7.5% by weight besides the at least 60% by weight phosphatidylcholine.

This application claims priority to German patent application number 10 2012 009 575.9, filed May 15, 2012, the contents of which are incorporated herein by reference.

The present invention relates to a pharmaceutical composition for the application in human and animals with the generic part of patent claim 1.

Pharmaceutical compositions for the application in human and animal, which contain at least one systemically and/or locally acting, topically applicable active ingredient, are already known for a long time.

For example the EP 0 704 206 A describes such a pharmaceutical composition, which is present as a liquid and is able to be topically sprayed on as droplets. Also such a pharmaceutical composition is known from the DE 10 2010 027 315, which is present as a liquid and is applied topically as a foam in human and animal, whereby both known compositions unanimously comprise a phospholipid, which contains phosphatidylcholine in a concentration of at least 60% by weight, referring to the total content of phospholipid. The oil content of this phospholipid is neither quantified in EP 0 704 206 nor in DE 10 2010 027 315 A. Although EP 0 704 206 mentions a concentration of 9% by weight of other not clarified common accompanying lipids for the there described phospholipidic gel forming agent, but it does not say how these accompanying lipids are determined, whereas DE 10 2010 027 315 A1 indeed describes oily components, but also does not explain how these oily components are analyzed quantitatively.

The object of the present invention is to provide a pharmaceutical composition of the stated art, which has an especially high ability for permeation of pharmaceutical active ingredients.

This object is solved according to the invention by a composition having the characteristics of patent claim 1.

The inventive pharmaceutical composition, which is used for the topically application in human and animal, contains at least one systemically and/or locally acting, topically applicable pharmaceutical active ingredient, which is consecutively referred to as active ingredient. Furthermore the inventive composition contains at least one phospholipid, which improves the transport of the active ingredient trough the cell membrane during a topical application of the inventive composition, whereby the phospholipid contains a concentration of phosphatidylcholine of at least 60% by weight referring to the total weight of the phospholipid (dry weight). The inventive pharmaceutical composition provides such a liquid consistency, that it is able to be sprayed on as droplets or fog (mist) or that it is able to be topically applicable as foam through appropriate customary applicators, for example through the applicator described in the DE 10 2010 027 315 A1, which is produced and distributed by the company Rexam/Airspray (www.rexamairspray.com) under the labeling “M3 Minischäumer” (M3 mini foamer) or through suitable applicators, which are produced and distributed by the company Calmar/MeadWestvaco (Keltec), whereby the content of the DE 10 2010 027 315 is added to the disclosure of the present application. According to the present invention, such a phospholipid is contained in the claimed composition, that has beside at least 60% by weight phosphatidylcholine an oil in a concentration of maximum 7.5% by weight, referring to the dry total weight of the phospholipid, too. Hereby this oil, which is included in the inventive composition in the phospholipid, is quantitatively determined according to the analytic methods, as they are described exactly subsequently at the beginning of the examples under the heading “quantitative determination of the oil contained in the phospholipids”.

The inventive composition shows a number of advantages. Thus it is firstly recorded that the inventive composition has a improved pharmaceutical efficiency, which is traced to the fact that an accelerated penetration and especially an accelerated permeation of the systemically and/or locally acting pharmaceutical active ingredient is brought about the selection of the special phospholipid described previously, which is characterized one the one hand by a minimum concentration of 60% by weight of phosphatidylcholine and on the other hand by a restricted concentration of oil of maximum 7.5% by weight, so that these active ingredients can reach the particular target site accordingly in higher concentrations and/or faster during topical application of the pharmaceutical composition. Furthermore it could be observed astonishingly that an improvement of the storage stability of the inventive composition is caused by the limitation of the oil concentration in the phospholipids present in the inventive composition, which is due to the fact, that such oils usually present in the phospholipids have a high concentration of unsaturated double bounds, which have a relatively high sensitivity to oxidation while storage in an air atmosphere. If such oxidation processes should run increasingly, according to the suggestions of the inventor of the present application they can lead to the fact that undesirable oxidation products can arise, which for their part catalyze and/or bring about the accelerated decay of the active ingredient and/or the phospholipid, which can cause a reduced storage stability and/or a reduced pharmaceutical efficiency. If the before described oxidation processes of the oil present in the phospholipid occur and if these products of the oil degradation bring about or catalyze the degradation of the phospholipid, thus already low traces of products of the phospholipid degradation lead to an unpleasant and undesirable development of odor, which becomes all the stronger, the longer the inventive composition is stored after the first use. This development of odor keeps the patient from using the inventive composition in the extent that is medically induced and/or necessary.

According to the inventor, the aforementioned accelerated penetration and permeation of the inventive composition is reduced to the fact that free fatty acids, sterols, mono- and diglycerides, triglycerides, tocopherols, and/or fatty acid esters as well as comparable substances are contained in the oil, which can influence the permeation and/or the penetration of the active ingredient in a negative way. Especially if the inventive composition contains such a phospholipid, which comprises a concentration of maximum 5.8% by weight of oil, the advantages described before in connection with the inventive composition are enhanced further.

An even more distinct improvement of the advantages arises in the inventive composition if it contains such a phospholipid, that has less than 4.8% by weight and especially between 2% by weight and 4.8% by weight of oil.

As it is described previously in the connection with the inventive composition, the inventive composition contains at least one phospholipid, whereby preferably a mixture of phospholipids is used, which contains lyso-phosphatidylcholine, phosphatidic acid, phosphatidylethanolamine and/or phosphatidylinositol besides phosphatidylcholine as main component.

Furthermore the term “and/or” used in the present description covers additively as well as alternatively the so linked single elements of an enumeration, so that these elements are seen linked optionally with “and” respectively with “or”. Furthermore the terms used in singular obviously include the plural, too.

Moreover it is recorded, that the term phospholipid used in the present description of course does not only cover a single phospholipid, but also a mixture of phospholipids, whereby the phospholipid respectively the phospholipid mixture can be of natural or synthetic origin.

Thus principally every phospholipid can be contained in the inventive composition, as far as this phospholipid has the aforementioned minimum concentration of at least 60% by weight phosphatidylcholine as well as the maximum concentration of oil described previously in connection with the inventive composition. Therefore also such embodiments of the inventive composition are covered by the present description, in which the phospholipid respectively phospholipid mixture present in the inventive composition is a synthetic phospholipid respectively a synthetic phospholipid mixture. Preferably the inventive pharmaceutical composition contains such a phospholipid respectively phospholipid mixture, which is isolated from vegetable components, especially from grain seeds and/or oil-rich seeds and preferably from soy beans or from sunflowers.

In another embodiment of the inventive pharmaceutical composition this contains such a phospholipid, whose concentration of phosphatidylcholine amounts at least 75% by weight, whereby a very particularly preferred arrangement of the inventive composition comprises such a phospholipid, in which the concentration of phosphatidylcholine amounts 78.1% by weight±3% by weight.

In the description of the inventive composition above it is repeatedly referred to the fact, that the phospholipid can also be a phospholipid mixture. Especially the phospholipid present in the inventive composition has a concentration of lyso-phosphatidylcholine of less than 5.6% by weight, preferably a concentration between 5.5% by weight and 1.5% by weight, a concentration of phosphatidylethanolamine of less than 5.2% by weight, preferably a concentration between 5.1% by weight and 2.3% by weight and a concentration of phosphatidic acid of less than 2.9% by weight, preferably a concentration between 2.5% by weight and 0.9% by weight, each referring to the total concentration of phospholipid. Moreover glycophospholipids, especially lyso-phospholipids, preferably in low concentrations, which means concentrations in the range of about 1% by weight up to 2% by weight, can be present in the inventive composition.

Preferably the inventive composition is available as a liquid composition before its application, which is applied as fog, droplets or foam. Hereby the inventive composition then contains an inorganic or organic solvent depending on the desired and/or on the viscosity required for the particular application, whereby water as inorganic solvent and a physiological harmless solvent as organic solvent is contained in the inventive composition.

Water in terms of the present description includes all aqueous systems, which are physiological harmless and legally admitted, and covers also such aqueous systems besides distilled water, de-ionized water and ultra-pure water, that contains respective buffer system for the correction of the pH-value or that contains also salts, especially common salt.

The inventive composition contains as preferred organic, physiological harmless solvent at least one alcohol, especially ethanol, isopropyl alcohol, one or more glycols and/or glycerol besides water or in addition to water.

Appropriate glycols, which are supposed to be the organic solvent in the inventive composition in special embodiments, are chosen from the group comprising propylene glycol, butylene glycol, pentylene glycol and hexylene glycol.

An additional embodiment of the inventive composition provides that this contains a solvent mixture made of water and at least one alcohol, preferably isopropyl alcohol.

In dependence on the desired and/or on the viscosity required for the particular application, the concentration of the inorganic and/or organic solvent varies in the inventive composition. In the liquid composition, the concentration of the water is between 50% by weight and 95% by weight, especially between 55% by weight and 75% by weight, and the concentration of the at least one organic solvent and preferably of the alcohol is between 5% by weight and 50% by weight, especially between 8% by weight and 25% by weight.

According to the at least one pharmaceutical active ingredient present in the inventive composition it is recorded that the pharmaceutical active ingredient is such one, which owns a local and/or systemical pharmaceutical efficiency during a topical application. The active ingredient present in the inventive composition is especially chosen from the group, which includes local anesthetics, anti-allergic agents, dermatics, active ingredients against flu infections and colds, active ingredients for the treatment of neuropathies, active ingredients for the treatment of disturbed circulation, chemotherapy drugs, quinine, antimycotics, antibiotics, thalidomide, serotonin, eicosanoids, analgesics, anticonvulsants, nonsteroidal antirrheumatics, leukotrienes, leukotriene inhibitors, androgens, antiandrogens, corticoids, opiate receptor antagonists, blood clotting inhibitory substances, thrombocyte aggregation inhibitors, histamine antagonists, regulatory and enzymatically acting peptides and proteins, nucleic acids (single and double-stranded DNA, single and double-stranded RNA, snRNA, DNA oligonucleotides, RNA oligonucleotides) and oligopeptides, antipruritics, antidiabetics, prostaglandins, prostaglandin synthesis inhibitors, antiviral-acting or virostatic-acting substances, antimicrobial-acting substances, active ingredients against prions, immune suppressants, hormones, active ingredients for treatment of warts or wounds, especially chronic wounds, vitamins, plant extracts or essences of plant extracts, psychoactive drugs, active ingredients which influences sleep, analeptics, general anesthetics, muscle relaxants, antiepileptics, antiparkinson agents, antiemetics, antiparasitics, ganglion-active ingredients, sympathetic-active ingredients, parasympathetic-active ingredients, antibacterial-acting drugs, calcium antagonists, cardiovascular agents, antiasthmatics, antitussives, expectorants, hepatics, diuretics, choleretics, disinfectants, trace elements, antiinfectives, cytostatics, antimetabolites, hormone antagonists, immune modulators, as well as derivates and salts of the aforementioned active ingredients.

In the inventive composition, the concentration of the active ingredient varies in dependence on the particular active ingredient and the kind of topical application and are between 0.01% by weight and 15% by weight referring to the total weight of the inventive composition.

Especially suitable embodiments, which are characterized by a particularly high pharmaceutical efficiency in a topical application, contain an analgesic as the at least one pharmaceutical active ingredient, which is chose from a group, consisting of diclofenac, ketoprofen and ibuprofen.

If diclofenac is chosen as active ingredient in the previously described embodiment, the inventive composition advantageously contains a derivate of diclofenac and preferably an alkali salt of diclofenac, which is especially present in the inventive composition as sodium salt.

With the topically applicable inventive action, that contains diclofenac as active ingredient, good results can be achieved by the fact that the diclofenac and especially the alkali salt of diclofenac and preferably the sodium salt of diclofenac is present in the composition in a concentration between 0.1% by weight and 10% by weight, preferably in a concentration between 1% by weight and 6% by weight and especially in a concentration between 2% by weight and 4% by weight.

However if another embodiment of the inventive composition contains ketoprofen as the at least one active ingredient, hereby in the inventive composition the concentration of the ketoprofen varies between 5% by weight and 15% by weight, preferably between 8% by weight and 12% by weight.

According to the concentration of phospholipid, which in contained in the inventive composition, it is generally recorded, that this concentration depends especially on the type of disease, which should be treated topically and/or systemically with the inventive composition and which kind of application is chosen, whether as fog, droplets or foam. Especially by variation of the concentration of the at least one phospholipid present in the inventive composition, also the viscosity of the liquid composition can be varied, so that according to this a desired size of the fog droplets or of the droplets can be configured or also the kind of foam can be varied by the concentration of the phospholipid. The inventive composition preferably contains the at least one phospholipid in a concentration between 0.5% by weight and 20% by weight, especially in a concentration between 5% by weight and 13% by weight.

A special variant of the previously described embodiments of the inventive composition, it further contains especially at least one complexing agent, preferably ethylene tetra acetic acid, at least one buffering agent, especially a phosphate buffer, at least one antioxidant, preferably palmitoyl ascorbic acid, at least one perfume and/or one aroma.

The term “topical” used in the present text includes every endemic outer application of the inventive composition, especially an application on top of the skin of human and animal. Hereby the term skin covers not only the respectively affected parts of the skin but also healthy parts of the skin as well as every available surfaces of the human and animal body, on which the inventive composition can be applied for the local application and/or the systemical application, besides the skin or scalp as such in particular also such as nails, hair, teeth, hooves or the mucous membrane of mouth, nose, vagina or foreskin, the parts of the ear and especially the parts of the inner ear, the part of the bowel outlet and the rectum, the part of the eyes, especially the part under the eyelid, as for example the conjunctiva, the cornea and the lachrymal sack.

Following, the inventive composition will be clarified by means of examples in connection with the figures, whereby

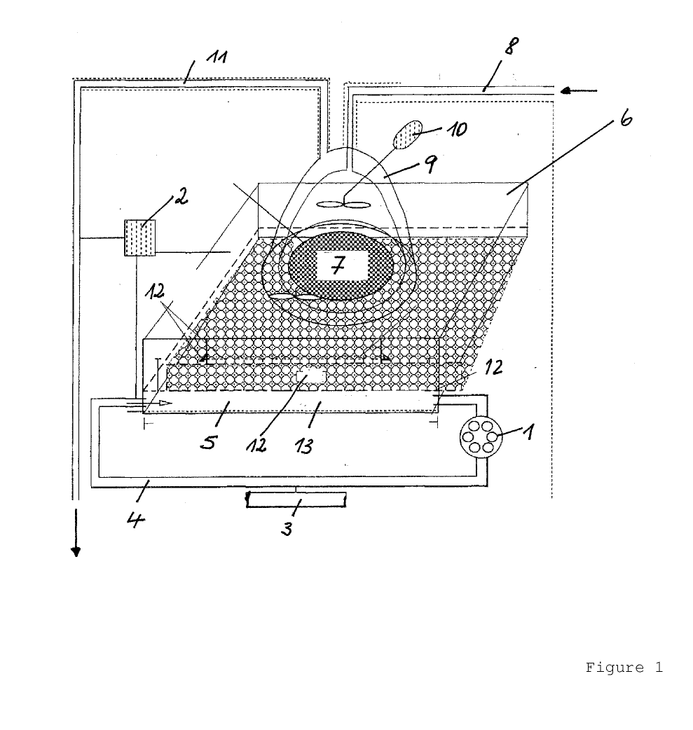

FIG. 1 shows an experimental setup for the determination of the permeation.

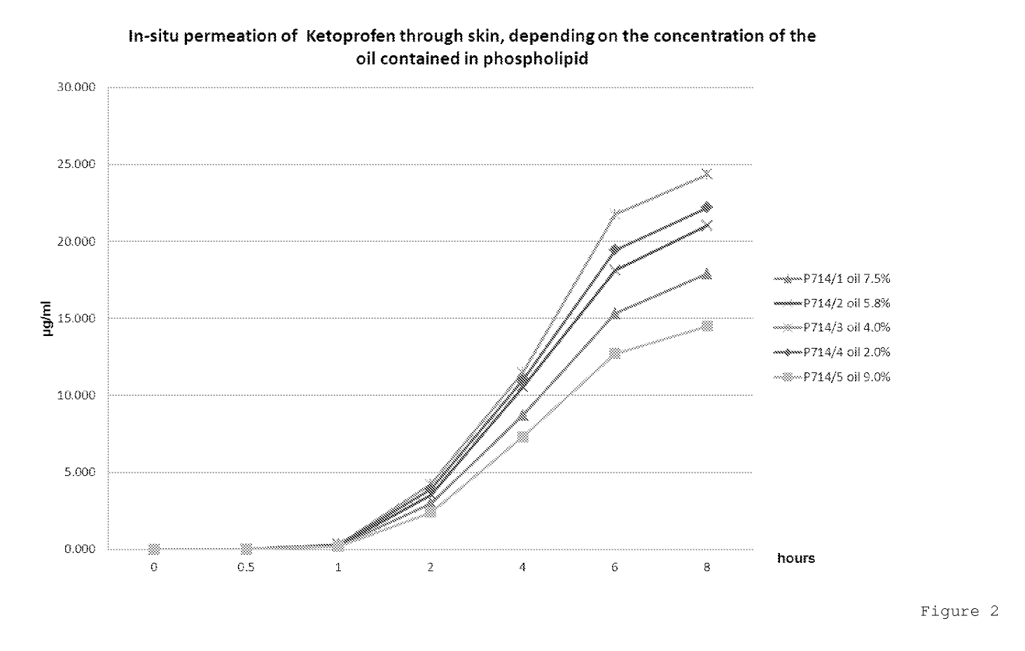

FIG. 2 shows a diagram of the permeation of ketoprofen as active ingredient depending on the concentration of the oil contained in phospholipid.

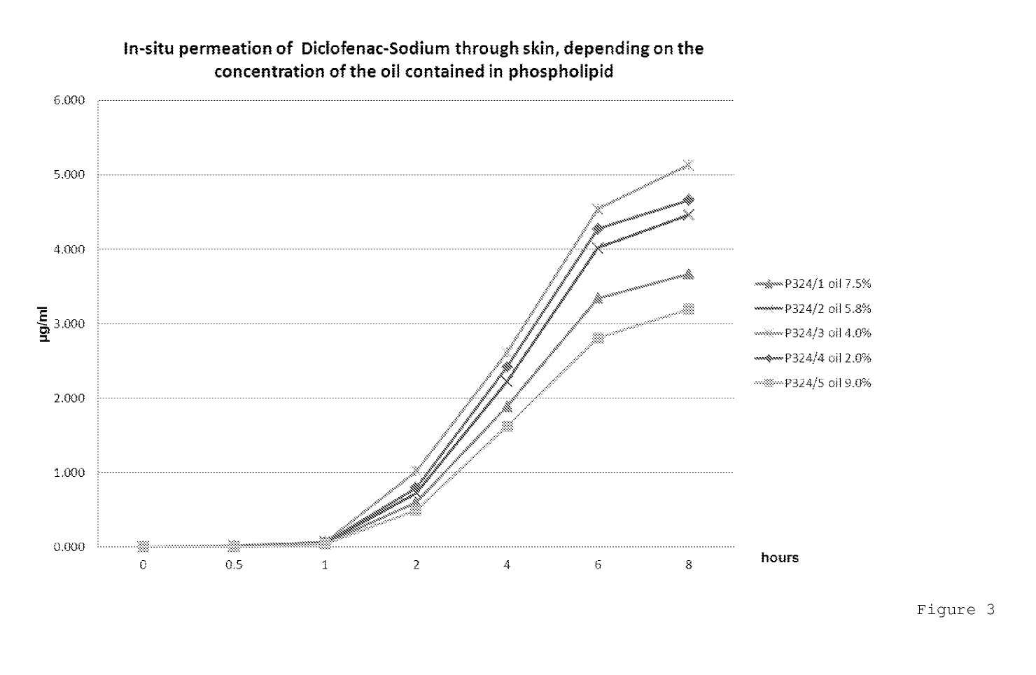

FIG. 3 shows a diagram of the permeation of diclofenac-sodium as active ingredient depending on the concentration of the oil contained in phospholipid.

### QUANTITATIVE DETERMINATION OF THE OIL CONTAINED IN THE PHOSPHOLIPID

The phospholipid, which should be analyzed, is weighed, solved in about 15 ml to 20 ml diethyl ether and separated quantitatively on a chromatography column, as it is described in the following, using about 130 ml to 150 ml diethyl ether as eluent. The whole ether eluate is collected in a previously weighed round bottom flask. Subsequently the collected diethyl ether eluate is distilled of in a rotation steamer under vacuum and the round bottom flask with the oils present therein is weighed again after air-conditioning in standard atmosphere, whereby the oil-free phospholipids are adsorbed in the column by the adsorbent and remain there. Out of this the percentage of the oil contained in the phospholipid is calculated as follows:

E=weight of sample taken of phospholipid base substance [g]

LK=dead weight of the round bottom flask [g]

R=backweight of the round bottom flask after distillation off the diethyl ether and storage under standard atmosphere [g]

Ö=percentage of oil in the phospholipid base substance

$$Ö = \frac{R - LK \times 100}{E} [ \% ]$$

The previously stated weight values in gram were determined on an analytical balance with an accuracy of 0.0001 g, whereby the phospholipid base substance, which should be analyzed, is weighed precisely in a magnitude of about 1 g.

For the preparation of the columns, it is preceded as follows:

The silica gel, used as adsorbent (silica gel 60, 0.063-0.2 mm, for example manufacturer: Merck, article number 7754) is initially adjusted to a constant water content of 14.3% by weight. For this purpose the water content of the particular used silica gel is determined by Karl-Fischer-titration in advance, and the missing amount of water for the obtaining of the water content of 14.3% by weight is added. From the so treated silica gel the water content is determined again by Karl-Fischer-titration, so that it is assured that the silica gel has a water content of 14.3% by weight.

30 g of the silica gel, which is adjusted to a water content of 14.3% by weight is suspended in diethyl ether and brought in the chromatography column, which has a diameter of 25 mm. The surplus ether is drained out of the column as far as an about 1 cm high ether layer stays above the adsorbent. On the so prepared column the sample, which should be analyzed, is applied and separated, like it is described at the beginning.

All solvents, used in this analyze, are present in p.a.-purity.

### Production of Phospholipid with Different Oil Content

The previously described analytical and gravimetric method for the determination of the quantitative oil content was modified in such a way, that the there described separation by column chromatography of the oil was now applied as preparative separation by column chromatography to produce the following described phospholipid, which are denoted with phospholipid 1 to 5, which differ in their oil content.

Therefore it was preceded as follows:

4,500 g of the silica gel, which is adjusted to a water content of 14.3% by weight is suspended in diethyl ether and brought in a preparative chromatography column, whereby the water content of the silica gel was adjusted and controlled like it is described previously in connection with the quantitative determination of the oil content of the phospholipid. The surplus ether is drained out of the column as far as an about 1 cm high ether layer stays above the absorbent. On the so prepared column the phospholipid sample, which should be preparative separated and which is solved in about 2.5 l diethyl ether (weight of sample taken about 150 g) is applied and separated, as it is also described initially in connection with the quantitative determination of the oil. The collected diethyl ether eluate is distilled off in vacuum thereby extracting the so isolated oil. Hereby about 140 g oil can be isolated.

The adsorbent, which is loaded with the oil-free phospholipid was removed out of the preparative column and the diethyl ether was carefully removed.

The so dried absorbent was extracted several times with a mixture of chloroform and methanol (2:1; V:V), whereby the extractions which contained the oil-free phospholipid, was combined. After the careful separation of the mixture of solvents the so isolated dry phospholipid was weighed after forming five, referring to the phospholipid similar concentrated samples and solved in ethanol. The oil extracted previously during the separation with diethyl ether was added to each of the five ethanolic phospholipid solutions in the given quantities (2% by weight, 4% by weight, 5.8% by weight, 7.5% by weight, 9% by weight), after this oil was previously solved in ethanol, too. After intensive mixing of every oil-free phospholipid with the oil, the ethanol was removed under formation of the following quantified phospholipid 1 to 5, whereby these phospholipids 1 to 5 were used for the production of the embodiments 1 to 10.

Thereby the five different phospholipid samples showed the following oil content;

Phospholipid 1, oil content 7.5% by weight

Phospholipid 2, oil content 5.8% by weight

Phospholipid 3, oil content 4% by weight

Phospholipid 4, oil content 2% by weight and

Phospholipid 5, oil content 9% by weight.

All five phospholipids (phospholipid 1 to 5) contains the concentration of

Phosphatidylcholine 76±3% by weight,

lyso-phosphatidylcholine≦6% by weight

phosphatidylamine≦6% by weight and

phosphatidic acid≦6% by weight,

whereby these data refers to the dry weight of the phospholipid.

Furthermore, all phospholipids showed an acid value of less than 10 and a peroxide value of less than 10, too.

The following examples 1 to 10 were produced under the use of the previously described phospholipids 1 to 5.

Congruently the examples 1 to 5 each contain 10% by weight of ketoprofen as pharmaceutical active ingredient and the examples 6 to 10 each contain 4% by weight of diclofenac sodium as pharmaceutical active ingredient.

Furthermore the examples 1 to 5 each contain 59.48% by weight of water, 10% by weight of propylene glycol, 8% by weight of isopropyl alcohol, 0.25% by weight of sodium dihydrogen phosphate, dihydrate, 0.57% by weight of disodium phosphate, dodeca hydrate, 1.55% by weight of sodium hydroxide solution and 0.15% by weight of peppermint oil.

The examples 1 to 5 only differ in the fact, that they indeed show identical amounts of the previously described and specified phospholipid, viz. 10% by weight, whereby it is bargained for the previously listed phospholipid 1 to 5, which differ in their concentration of oil as follows:

Example 1 contains 10% by weight of phospholipid 1 (oil content 7.5% by weight)

Example 2 contains 10% by weight of phospholipid 2 (oil content 5.8% by weight)

Example 3 contains 10% by weight of phospholipid 3 (oil content 4% by weight)

Example 4 contains 10% by weight of phospholipid 4 (oil content 2% by weight)

Example 5 contains 10% by weight of phospholipid 5 (oil content 9% by weight)

The previously listed 10% by weight of the phospholipids 1 to 5 each contain 7.5% by weight of phospholipid 1 to 5 and 2.5% by weight of absolute ethanol.

Moreover the examples 6 to 10 each contain 56.38% by weight of water, 15% by weight of propylene glycol, 10.25% by weight of isopropyl alcohol, 0.12% by weight of sodium dihydrogen phosphate, dihydrate, 0.20% by weight of peppermint oil, 0.66% by weight of disodium phosphate, dodeca hydrate, 0.04% by weight of disodium edetate, 0.02% by weight of palmitoyl ascorbic acid.

The examples 6 to 10 only differ in the fact, that they indeed show identical amount of the preciously described phospholipid, viz. 13.33% by weight, whereby it is bargained for the previously listed phospholipid 1 to 5, which differ in their oil concentration:

Example 6 contains 13.33% by weight of phospholipid 1 (oil content 7.5% by weight)

Example 7 contains 13.33% by weight of phospholipid 2 (oil content 5.8% by weight)

Example 8 contains 13.33% by weight of phospholipid 3 (oil content 4% by weight)

Example 9 contains 13.33% by weight of phospholipid 4 (oil content 2% by weight)

Example 10 contains 13.33% by weight of phospholipid 5 (oil content 9% by weight)

The previously listed 13.33% by weight of the phospholipids 1 to 5 each contain 9.998% by weight of phospholipid 1 to 5 and 3.332% by weight of absolute ethanol.

For all examples 1 to 5, that contain ketoprofen as active ingredient, an identical manufacturing process was used. Therefore ketoprofen, propylene glycol and isopropyl alcohol were mixed in a mixing container, whereby this mixing process was run in the presence of nitrogen or argon.

About 90% by weight of the amount of sodium dihydrogen phosphate, as well as the disodium phosphate and the sodium hydroxide solution were mixed together.

The previously firstly produced mixture was added with the particular phospholipid (phospholipid 1 to 5) with a temperature between 20° C. and 25° C. and afterwards mixed so long until a clear, yellowish solution resulted. Then the previously mixed buffer solution, which is described previously as second, was added to this yellowish clear solution, whereby the solutions were mixed with a temperature of maximum 30° C. (in the presence of argon or nitrogen) until a homogeneous solution resulted, to which the remaining buffer solution and the peppermint oil was added and whose pH-value was adjusted to a value between 7.3 and 7.5.

For all examples 6 to 10 containing diclofenac-sodium as active ingredient, an identical manufacturing process was used. Therefore about 90% by weight of the water (purified water) was mixed with the disodium phosphate, sodium dihydrogen phosphate and the sodium-edetate under a temperature between 25° C. and 30° C. and a number of revolutions of 600 revolutions per minute. The so prepared solution was cooled to 20° C. and 25° C.

In a second mixing container the propylene glycol, the isopropyl alcohol and the particular phospholipid (phospholipid 1 to 5) were mixed under careful stirring, until it became a homogeneous solution. The ascorbylpalmitate and the diclofenac-sodium were added to this mixture under maintenance of the stirring. The so prepared homogeneous solution was mixed until a clear solution resulted, which was added with peppermint oil under constant stirring. After measurement of the pH-value, this was adjusted to a value between pH 7.4-7.6 trough addition of sodium hydroxide or diluted hydrochloric acid. During the production the temperature was kept constantly between 20° C. and 30° C.

### Permeation Study of the Previously Described Examples 1 to 10

The consecutively described in situ-model shown in FIG. 1 is especially suitable for the comparative determination of the rate of permeation. The experience of many in vivo-experiments on young pigs and on test persons underlies this model. It was developed in consideration of the most relevant in-vivo conditions and could already be approved several times, so that it was validated with corresponding in-vivo studies with test persons.

In the experimental setup for the determination of the permeation, shown in FIG. 1, 90 ml of the acceptor medium 13, which is covered underneath the skin sample that should be examined with a metal grille 12, is tempered to 35° C. and transferred continuously through pump 1 with 1000 ml/h. The permeation chamber 5, which is filled with the acceptor medium 13 has a volume of 50 ml, the compensation vessel 3 for the sampling and the tubes 4 has a total volume of 40 ml. The lumen can be filled with various, skin-physiological solutions. Authoritative for the choice of the acceptor medium is the solubility of the active ingredient and its verification.

For the active ingredient ketoprofen a phosphate buffer solution with pH 7.4 and for the active ingredient diclofenac-sodium a phosphate buffer solution with pH 8.0 was chosen. The choice of the acceptor medium takes into consideration of the setting conditions for the particular active ingredient.

The chamber 5 was filled free of bubbles, so that a complete and steady undercutting of the tissue can happen. The area of the application 7 amounts to 28.3 cm². The sampling was made intermittently through manual removal from the circulating medium and following automatically collection of the samples for the HPLC. The duration of the experiment was limited to 8 hours, whereby particular suitable samples of the acceptor medium were taken and analyzed at the beginning, after 30 minutes, one hour, two hours, four hours, six hours and 8 hours.

Due to the positioning of the air flow, the flow velocity of the induced, tempered air and the turbulence on the skin area the stratum corneum is not hydrated in the used experimental setup, which is shown in FIG. 1. An air circulation conduced therefore, which provides for an air supply of 300 ml air/minute at 23° C. through a circuit 8, a feeding in a hood 9, which is located above the application area 7 and in which a ventilator 10 ensures an air turbulence, and return air circuits 11 for the return of the air.

The particular skin sample, which should be examined, was applied on the metal grille 12, covered with the hood 9 (without occlusion), circulated with the air flow, which is swirled by the ventilator 10. The skin sample applied on the metal grille 12 is impinged with the particular composition, whereby the method of measurement for the determination of the permeation is described consecutively. For keeping the air temperature and the temperature of the acceptor medium constantly, the temperature control 2, which is only designated schematically, is used, as a hood 6 covers the measuring arrangement especially for the prevention of pollutions.

The in situ experiments were executed with excorporated human abdominal skin from different donors. The donors were between 40 and 50 years old. The studies were carried out with surgically removed, abdominal human skin. The size of the skin flaps, which were available, varied. That implies the execution of four tests per human skin flap.

The application area available for the application amounted for 28.3 cm². The skin was taken fresh, transported dryly and chilled to 4° C. After 2 hours at the latest the permeation experiment was initiated. By peeling of the subcutaneous fatty tissue the skin was prepared on a peeling block having a deep adjusting and inspected for their impeccable state by means of a leak test in the chamber with the medium by a pressure flushing before and after the examination and microscopically. The thickness of the skin (without fatty tissue) amounts for 1 mm±0.1 mm. The macroscopic unharmed skin samples, which were proved for tightness, were directly transferred to the experiment apparatus, whereby the skin samples fixed on a grille and the available area were conditioned without occlusion. The conditioning was achieved by the regulation of the pre-heating temperature and the tempering of the air tubes, as well as by the velocity of the air flow, which skims over the sample area. These parameters were kept constantly within one test series. The skin sample was undermined steadily circulating by the phosphate buffer. The temperature of the skin sample was checked by probes and the humidity was checked by the corneometer. The supply of the skin sample took place by the phosphate buffer. A possible contamination of the skin with the disinfectant phenylmercuroborate was considered in the analytics.

The liquid composition was distributed evenly on the application area of 28.3 cm² with a microdispenser (round pistill). Five minutes after the first application, a distribution of the applied composition on the skin sample was made again.

At the application of up to 1 g composition per 28.3 cm² application area no surplus of the particular composition on the skin surface in the range of the application area was noticeable.

The permeation profile of ketoprofen and diclofenac-sodium were made by the taking of duplicates samples after 0, 0.5, 1, 2, 4, 6 and 8 hours. The determination of the ketoprofen and the diclofenac-sodium occurs following directly after the sampling through an auto sampler. For the evaluation of the permeation of the particular active ingredient its concentration in the acceptor medium was determined.

Therefore the concentration of the ketoprofen was determined by high pressure chromatography (HPLC-column, Xterra RP 18 5 μm, 4.6×150 mm—mode: isocratic—flow: 1.0 mL·min-1, flow substance: H₂O/CAN/KH₂PO₄, pH of the buffer 3.5 (55:43:2 (v/v/v)), buffer: p.a. chemicals+deionized water; flow substance not degassed) by using the UV-detector (UV-detector Waters Alliance 2795 with detector Waters 2487 dual wavelength 254 nm).

The determination of the concentration of the diclofenac-sodium also occurs by high pressure chromatography by using an UV-detector (HPLC-system with the components as follows: HPLC-pump Merck/Hitachi L-6200, (ternary/low-pressure gradient), UV-detector Merck/Hitachi L-4000, double beam instrument, measurement range 195-380 nm, Integrator Merck/Hitachi D-2500, HPLC-column: LiChrospher 100 (Merck), RP18 (5 u·m), 250 mm length, LiChroCART-cartidge system, flow substance: methanol/citrate buffer (3/1); flow substance not degassed).

The results of the previously described permeation study for the active ingredient ketoprofen are summarized in the following table 1 and in the relating FIG. 2 and the results for the active ingredient diclofenac-sodium are summarized in the following table 2 and the relating FIG. 3.

In all examinations always the same, previously stated amount of the composition was applied on the skin sample, which correspond to a concentration of the active ingredient of about 25 mg ketoprofen, respectively of about 10 mg diclofenac-sodium. The reported values particularly correspond to the average value of four tests.

TABLE 1
Permeation of ketoprofen in the acceptor medium
Concentration (μg) ketoprofen per volume unit (ml) of the acceptor medium

| | example | | | | |
|---|---|---|---|---|---|
| Oil | 1 | 2 | 3 | 4 | 5 |
| concentration | 7.5% by | 5.8% by | 4.0% by | 2.0% by | 9.0% by |
| removal time | weight | weight | weight | weight | weight |
| in h | Concentration in μg/ml | | | | |
| 0 | 0.000 | 0.000 | 0.000 | 0.000 | 0.000 |
| 0.5 | 0.008 | 0.011 | 0.013 | 0.013 | 0.002 |
| 1 | 0.239 | 0.274 | 0.310 | 0.303 | 0.195 |
| 2 | 2.987 | 3.524 | 4.241 | 3.912 | 2.389 |
| 4 | 8.726 | 10.558 | 11.518 | 11.021 | 7.329 |
| 6 | 15.309 | 18.112 | 21.738 | 19.442 | 12.706 |
| 8 | 17.901 | 21.018 | 24.345 | 22.182 | 14.499 |

TABLE 2
Permeation of diclofenac-sodium in the acceptor medium
Concentration (μg) diclofenac-sodium
per volume unit (ml) of the acceptor medium

| | example | | | | |
|---|---|---|---|---|---|
| Oil | 6 | 7 | 8 | 9 | 10 |
| concentration | 7.5% by | 5.8% by | 4.0% by | 2.0% by | 9.0% by |
| removal time | weight | weight | weight | weight | weight |
| in h | Concentration in μg/ml | | | | |
| 0 | 0.000 | 0.000 | 0.000 | 0.000 | 0.000 |
| 0.5 | 0.000 | 0.009 | 0.015 | 0.011 | 0.000 |
| 1 | 0.052 | 0.061 | 0.073 | 0.066 | 0.042 |
| 2 | 0.601 | 0.726 | 1.019 | 0.799 | 0.492 |
| 4 | 1.890 | 2.223 | 2.613 | 2.417 | 1.625 |
| 6 | 3.343 | 4.019 | 4.542 | 4.273 | 2.808 |
| 8 | 3.669 | 4.462 | 5.133 | 4.659 | 3.192 |

We claim:

1. A pharmaceutical composition for application in human and animals, with at least one systemically and/or locally acting, topically applicable active ingredient and with at least one phospholipid, which improves the transport of the active ingredient trough the cell membrane and which contains a concentration of at least 60% by weight phosphatidylcholine, referring to the phospholipid, whereby the composition has such a liquid consistency, that it is able to be sprayed as droplets or as a foam, wherein the composition contains such a phospholipid, that additionally comprises oil in a concentration of maximum 7.5% by weight besides the at least 60% by weight phosphatidylcholine.

2. The pharmaceutical composition according to claim 1, wherein the composition comprises such a phospholipid, that contains oil in a concentration of maximum 5.8% by weight.

3. The pharmaceutical composition according to claim 1, wherein the composition contains such a phospholipid, that comprises oil in a concentration of less than 4.8% by weight, especially in a concentration between 2% by weight and 4.8% by weight.

4. The pharmaceutical composition according to claim 1, wherein the phospholipid is a phospholipid mixture contains lyso-phosphatidylcholine, phosphatidic acid, phosphatidylethanolamine and/or phosphatidylinositol besides phosphatidylcholine.

5. The pharmaceutical composition according claim 1, wherein the phospholipid is a phospholipid, which is isolated from vegetable components, especially from grain seeds and/or oil-rich seeds and preferably from soy beans or from sunflowers.

6. The pharmaceutical composition according to claim 1, wherein the phospholipid contains a concentration of phosphatidylcholine of at least 75% by weight.

7. The pharmaceutical composition according to claim 1, wherein the phospholipid contains a concentration of phosphatidylcholine of 78.1% by weight±3% by weight.

8. The pharmaceutical composition according to claim 7, wherein the phospholipid contains a concentration of lyso-phosphatidylcholine of less than 5.6% by weight, of phosphatidylethanolamine of less than 5.2% by weight and of phosphatidic acid of less than 2.9% by weight.

9. The pharmaceutical composition according to claim 8, wherein the phospholipid contains the lyso-phosphatidylcholine in a concentration between 5.5% by weight and 1.5% by weight, the phosphatidylethanolamine in a concentration between 5.1% by weight and 2.3% by weight and the phosphatidic acid in a concentration between 2.5% by weight and 0.9% by weight.

10. The pharmaceutical composition according to claim 1, wherein the composition contains an inorganic or organic solvent.

11. The pharmaceutical composition according to claim 10, wherein the composition contains water and/or an alcohol, especially ethanol, isopropyl alcohol one or several glycols and/or glycerol as solvent.

12. The pharmaceutical composition according to claim 11, wherein the composition contains propylene glycol, butylene glycol, pentylene glycol, and/or hexylene glycol as glycol.

13. The pharmaceutical composition according to claim 1, wherein the composition contains a solvent mixture comprising water and at least one alcohol, preferably isopropyl alcohol.

14. The pharmaceutical composition according to claim 13, wherein the concentration of water in the liquid composition varies between 50% by weight and 95% by weight, preferably between 55% by weight and 75% by weight, and wherein the concentration of the at least one alcohol varies between 5% by weight and 50% by weight, preferably between 8% by weight and 25% by weight.

15. The pharmaceutical composition according to claim 1, wherein the at least one active ingredient is selected from the group consisting of local anesthetics, anti-allergic agents, dermatics, active ingredients against flu infections and colds, active ingredients for the treatment of neuropathies, active ingredients for the treatment of disturbed circulation, chemotherapy drugs, quinine, antimycotics, antibiotics, thalidomide, serotonin, eicosanoids, analgesics, anticonvulsants, nonsteroidal antirrheumatics, leukotrienes, leukotriene inhibitors, androgens, antiandrogens, corticoids, opiate receptor antagonists, blood clotting inhibitory substances, thrombocyte aggregation inhibitors, histamine antagonists, regulatory and enzymatically acting peptides and proteins, nucleic acids (single and double-stranded DNA, single and double-stranded RNA, snRNA, DNA oligonucleotides, RNA oligonucleotides) and oligopeptides, antipruritics, antidiabetics, prostaglandins, prostaglandin synthesis inhibitors, antiviral-acting or virostatic-acting substances, antimicrobial-acting substances, active ingredients against prions, immune suppressants, hormones, active ingredients for treatment of warts or wounds, especially chronic wounds, vitamins, plant extracts or essences of plant extracts, psychoactive drugs, active ingredients which influences sleep, analeptics, general anesthetics, muscle relaxants, antiepileptics, antiparkinson agents, antiemetics, antiparasitics, ganglion-active ingredients, sympathetic-active ingredients, parasympathetic-active ingredients, antibacterial-acting drugs, calcium antagonists, cardiovascular agents, antiasthmatics, antitussives, expectorants, hepatics, diuretics, choleretics, disinfectants, trace elements, antiinfectives, cytostatics, antimetabolites, hormone antagonists, immune modulators, and derivates and salts of the aforementioned active ingredients.

16. The pharmaceutical composition according to claim 15, wherein the analgesic is selected from the group consisting of diclofenac, ketoprofen and ibuprofen.

17. The pharmaceutical composition according to claim 15, wherein in the composition the analgesic is diclofenac and the diclofenac is present in the composition as alkaline salt, especially as sodium salt.

18. The pharmaceutical composition according to claim 16, wherein the diclofenac is contained in the composition in a concentration between 0.1% by weight and 10% by weight, preferably in a concentration between 1% by weight and 6% by weight.

19. The pharmaceutical composition according to claim 15, wherein the composition contains ketoprofen as at least one active ingredient and wherein the concentration of the ketoprofen in the composition varies between 5% by weight and 15% by weight, preferably between 8% by weight and 12% by weight.

20. The pharmaceutical composition according to claim 1, wherein the composition comprises at least one phospholipid in a concentration between 0.5% by weight and 20% by weight, preferably in a concentration between 5% by weight and 13% by weight.

21. The pharmaceutical composition according to claim 1, wherein the composition furthermore contains a complexing agent, especially ethylene tetra acetic acid, at least one buffering agent, especially a phosphate buffer, at least one antioxidant, especially palmitoyl ascorbic acid, at least one perfume and/or an aroma.
